(19) United States
(12) Patent Application Publication
(10) Pub. No.: US 2026/0206796 A1
(43) Pub. Date: Jul. 23, 2026

(54) FEED COMPOSITIONS

(71) Applicant: Solugen, Inc., Houston, TX (US)

(72) Inventors: JiHye Kim, Sugar Land, TX (US); Jun Su An, Houston, TX (US); LoongYi Tan, Bellaire, TX (US); Kimchi Phan, Katy, TX (US); Catherine Gonzalez, Houston, TX (US); Darlen Galicia, Houston, TX (US)

(73) Assignee: Solugen, Inc., Houston, TX (US)

(21) Appl. No.: 19/144,814

(22) PCT Filed: Feb. 19, 2024
(86) PCT No.: PCT/US2024/016393

§ 371 Date: Jun. 30, 2025

Related U.S. Application Data
(60) Provisional application No. 63/446,663, filed on Feb. 17, 2023.

(51) Int. Cl. A23K 20/163; A23K 20/158; A23K 20/174; A23K 20/26; A23K 50/80; A61K 31/07; A61K 31/191; A61K 31/194; A61K 31/355; A61K 31/455; A61K 31/593; A61K 31/714; A61K 33/04; A61K 33/06; A61K 35/00; A61K 35/742; A61K 35/744; A61K 35/745; A61K 47/46; A61P 1/14; A61P 3/02

(52) U.S. Cl. CPC A23K 20/163; A23K 20/158; A23K 20/174; A23K 20/26; A23K 50/80; A61K 31/07; A61K 31/191; A61K 31/194; A61K 31/355; A61K 31/455; A61K 31/593; A61K 31/714; A61K 33/04; A61K 33/06; A61K 35/742; A61K 35/744; A61K 35/745; A61K 47/46; A61P 1/14; A61P 3/02; A61K 2035/115

(57) ABSTRACT

A composition includes a sugar oxidation product. In addition, the composition includes at least one material selected from the group consisting of oxides, nitrates, nitrites, sulfates, minerals, proteins, amino acids, macronutrients, and micronutrients.

### CROSS-REFERENCE TO RELATED APPLICATIONS

This application is a 35 U.S.C. § 371 national stage application of PCT/US 2024/016393 filed Feb. 19, 2024 and entitled “Feed Compositions,” which claims priority to U.S. Provisional Application No. 63/446,663 filed Feb. 17, 2023 entitled “Fish Feed Compositions,” each of which is hereby incorporated herein by reference in its entirety for all purposes.

### STATEMENT REGARDING FEDERALLY SPONSORED

### RESEARCH OR DEVELOPMENT

Not applicable.

### FIELD

The present disclosure relates generally to feed compositions. More particularly, the present disclosure relates to feed compositions with increased nutritional value and environmental benefit.

### BACKGROUND

Aquaculture is the fastest growing sector in agriculture due to the fact that world fisheries have reached a plateau while the demand for fish is growing. Fish utilize protein for energy purposes and therefore compared to terrestrial animal feeds, fish feeds are rich in proteins, often amounting to 40-50% of the feed. Fishmeal, a common source of protein in fish feeds, is becoming a rare and expensive commodity. Additionally, nitrogenous products (ammonia, nitrite and nitrate) and phosphorous that may be present in fishmeal, stemming from leftover food or partially digested protein and food, are harmful to the fish themselves, for example, potentially leading to growth retardation and at high levels cause mortality.

Additionally, phosphate discharge from fecal matter of the animals to the environment increases as the amount of algae bloom covering the ocean increases resulting in detrimental changes such as limited oxygen and nutrition source available to fish and aquatic species. Considering the potential impact of phosphate on aquatic areas, environmental regulations are becoming more stringent about phosphate level in the wastewater discharged from aquaculture farming.

Another challenge in aquaculture is disease outbreaks that have caused substantial economic losses around the world. In particular, the recent “early mortality syndrome” (EMS) due to plasmids in Vibrio bacteria is a growing threat. In addition to decreasing immunity, EMS causes substantial hepatopancreatic damage to shrimp and other fish species, which gradually induces mass mortalities. Moreover, with a greater restriction or altogether ban on antibiotic use in aquaculture farming, this can leave shrimp farmers with fewer options to protect their animals against bacterial diseases.

One alternative to the use of antibiotics is the inclusion of acidifiers in animal feed. Acidifiers may function as preservatives, exert defensive effects against microorganisms such as bacteria, fungus or mold which can result in the diminution of pathogen intake, improvements in feed digestion, nutrient digestibility and eubiosis in gut flora. Thus, an ongoing need exists for improved compositions and methods for addressing the challenges found in aquaculture.

### BRIEF DESCRIPTION OF DRAWINGS

For a detailed description of the aspects of the disclosed processes and systems, reference will now be made to the accompanying drawings in which:

FIG. 1a is a graph depicting the phosphate level (ppm) in water containing shrimp fed the indicated diets;

FIG. 1b is a graph depicting the percent phosphate reduction for shrimp fed a composition of the present disclosure compared to the control;

FIG. 2 is a graph of the shrimp survival rate (%) over the course of a 10 week feeding trial;

FIG. 3a is a bar graph of the mean (±SE) weight gain (%) and FIG. 3b is the feed conversion ratio (FCR) for tilapia fed a soybean meal-based diet (control), a soybean meal-based diet with a sugar oxidation product of the present disclosure, or a soybean meal-based diet with sodium citrate;

FIG. 4 is a bar graph of the mean (±SE) phosphorus levels (mg/l) in the water after tilapia were transferred to new water for 2 or 4 hours and previously fed a soybean meal-based diet (control), a soybean meal-based diet with a sugar oxidation product of the present disclosure or a soybean meal-based diet with sodium citrate;

FIG. 5 is a bar graph of the mean (±standard error) fecal phosphorus (mg/g dry weight) in tilapia's fecal matter fed a soybean meal-based diet (control), a soybean meal-based diet with a SOP of the present disclosure or a soybean meal-based diet with sodium citrate after two months feeding the diets. Different letters indicate significant differences (p<0.05);

FIG. 6 are slides depicting liver histology of tilapia fed (a) a soybean meal-based diet, (b) a soybean meal-based diet with a sugar oxidation product of the present disclosure, or (c) a soybean meal-based diet with sodium citrate (c, d and e). Livers in tilapia fed control and diet containing sugar oxidation product showed normal sinusoid organization with clear central veins and intact and well dispersed pancreatic islets (pi). However, tilapia fed with sodium citrate had varying degrees of hepatic lesions that included white blood cell (wbc) infiltrations and presence of lipofuscin-like material (lip) that are consistent with inflammation as well as more severe pathologies that included necrosis (nec) leading to the presence of plasma throughout the liver, hemorrhaging (hem) and extensive vacuolization (v). ×10 magnification (b, c, d, e) and ×20 magnification (d). Hematoxylin and eosin staining; and FIG. 7 are slides of the intestinal histomorphology in tilapia fed (a) a soybean meal-based diet (control), (b) a soybean meal-based diet with a sugar oxidation product of the type disclosed herein, or (c) a soybean meal-based diet with sodium citrate. The livers in tilapia fed citrate diet showed severe inflammation in goblet cell (gc) and the lamina propria (lp) at 20 magnification; hematoxylin and eosin staining.

### DETAILED DESCRIPTION

The following discussion is directed to various exemplary embodiments. However, one skilled in the art will understand that the examples disclosed herein have broad application, and that the discussion of any embodiment is meant only to be exemplary of that embodiment, and not intended to suggest that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc. ; greater than 0.10 includes 0.11, 0.12, 0.13, etc.).

In the following discussion and in the claims, the terms “including” and “comprising” are used in an open-ended fashion, and thus should be interpreted to mean “including, but not limited to . . . . ” As used herein, the phrases “consist(s) of” and “consisting of” are used to refer to exclusive components of a composition, meaning only those expressly recited components are included in the composition; whereas the phrases “consist(s) essentially of” and “consisting essentially of” are used to refer to the primary components of a composition, meaning that only small or trace amounts of components other than the expressly recited components (e.g., impurities, byproducts, etc.) may be included in the composition. For example, a composition consisting of X and Y refers to a composition that only includes X and Y, and thus, does not include any other components ; and a composition consisting essentially of X and Y refers to a composition that primarily comprises X and Y, but may include small or trace amounts of components other than X and Y. In embodiments described herein, any such small or trace amounts of components other than those expressly recited following the phrase “consist(s) essentially of” or “consisting essentially of” preferably represent less than 5.0 wt% of the composition, more preferably less than 4.0 wt% of the composition, even more preferably less than 3.0 wt% of the composition, and still more preferably less than 1.0 wt% of the composition. Use of broader terms such as comprises, includes, having, etc. should be understood to provide support for narrower terms such as consisting of, consisting essentially of, comprised substantially of, etc. Use of the term “optionally” with respect to any element of a claim is intended to mean that the subject element is required, or alternatively, is not required. Both alternatives are intended to be within the scope of the claim.

Disclosed herein are additives and additive compositions for use in feed such as fish feeds. These additives may be effective to increase the number of acidic species in the compositions to which they are included. Collectively, these additive compositions are termed feed-improving additives and designated FIAs. In one or more aspects, an FIA of the type disclosed herein is included in a feed. Herein, the term “feed” refers to any raw or processed product given to domesticated animals including complete feed, concentrated feed, feed additives and traditional feed.

In one or more aspects, the FIA is included in the feed composition for a domesticated or partially-domesticated animals for use as a food source and animals raised for use as a food source. Nonlimiting examples of animals domesticated or partially-domesticated for use as a food source include members of the genera bovinae, cervidae, lagomorphs, oves, and suidae. Other examples of animals domesticated or partially-domesticated for use as food source include birds, fish, crustaceans, and mollusks. In other aspects, the FIA is included in animal feed for animals not used as a food source, for example, in the feed for companion animals such as dogs and cats. In one or more aspects, the compositions disclosed herein are used to confer the disclosed benefits to any type of animal feed.

In one or more aspects, the FIA is included in a fish feed composition. Fish feed refers to a combination of ingredients (e.g., vitamins, protein) which results in a mixture containing levels of essential nutrients that meet or exceed the minimum requirements of the fish. It is contemplated that the FIA may be used in the feeding any animal and confer benefits thereto. It is contemplated that the FIA may be used in the feeding any animal and confer benefits thereto.

Herein, a FIA suitable for use in the present disclosure comprises one or more sugar oxidation products. In some aspects, the one or more sugar oxidation products comprises aldaric acid, uronic acid, glucaric acid, gluconic acid, glucuronic acid, glucose oxidation products, gluconic acid oxidation products, disaccharides, oxidized disaccharides, n-keto-acids, C2-C6 diacids, galactonic acid, galactaric acid, glutamic acid, glucodialdose, 2-ketoglucose, glucodiamine, glycolaldehyde, glyoxal, salts thereof, lactones thereof, or combinations thereof. In such aspects, the one or more sugar oxidation products may comprise less than about 5 wt. % maltose, maltotriose, fructose, higher molecular weight polysaccharides, oxidation products thereof, or combinations thereof based on the total weight of the one or more sugar oxidation products.

In one or more aspects, the one or more sugar oxidation products is a salt comprising a countercation. Countercations suitable for use in the disclosure include, but are not limited to zinc, iron, calcium, magnesium, manganese, silicates, borates, aluminates, aluminum, ammonium ion, sodium, potassium, cesium, strontium, an alkali metal, an alkaline earth metal or a combination thereof. In one or more aspects, the counter cation comprises potassium, sodium, aluminum, ammonium, magnesium, iron or combinations thereof. In one or more aspects, the one or more sugar oxidation products is the obtained from reacting glucose in the presence of an oxidizing agent with one or more biocatalysts (e.g., galactose oxidase or mutant thereof).

In one or more aspects, an FIA is included in a feed composition provided to fish and/or mammals as a source of nutrition. In one or more aspects, an FIA of the type disclosed herein is introduced to a fish and/or mammal feed that comprises oxides, nitrates, nitrites, sulfates, other minerals, macronutrients, and micronutrients.

In one or more aspects, a fish feed composition may comprise the 10 essential amino acids are methionine, arginine, threonine, tryptophan, histidine, isoleucine, lysine, leucine, valine, and phenylalanine. In one or more aspects, the protein in the fish feed is a plant-derived protein (e.g., soybean meal).

In one or more aspects, the fish feed comprises lipids. Nonlimiting examples of lipids suitable for use in the fish feed include fatty acids of the omega-3 and -6 (n-3 and n-6) families. Fatty acids used in the fish feed can be (a) saturated fatty acids (no double bonds), (b) polyunsaturated fatty acids (>2 double bonds), or (c) highly unsaturated fatty acids (>4 double bonds). In one or more aspects, the lipids in the fish feed comprise marine fish oil, algal oils or combinations thereof.

In one or more aspects, the fish feed composition comprises carbohydrates which include starches and sugars such as are available from grain. In one or more aspects, the fish feed composition comprises vitamins such as B vitamins (thiamine, riboflavin, niacin, pantothenic acid, pyridoxine, biotin, folic acid, and cobalamins), inositol, choline, and vitamin C (ascorbic acid)l vitamins A (retinol, betacarotene), D (cholecalciferol), E (tocopherols), and K (phylloquinone).

In one or more aspects, the fish feed comprises minerals such as calcium, sodium, chloride, potassium, chlorine, sulfur, phosphorous, magnesium, iron, copper, chromium, iodine, manganese, zinc, selenium or combinations thereof

In one or more aspects, an FIA may be included in mammal feed comprising one or more of the following ingredients: alfalfa meal and hay, Bermuda coastal grass hay, com plant, soybean hay, barley, com, oats, rice, sorghum, wheat, canola meal, cottonseed cakes and meals, peanut meal, safflower meal, and soybean (organic and genetically modified) feed and meal, distillers products, brewers dried grains, com gluten, sorghum germ cake and meal, peanut skins, wheat bran, dried citrus pulp, apple pomace, pectin pulp, beet, citrus, starch, cane molasses, meat meal, meat meal tankage, meat and bone meal, poultry meal, animal by-product meal, dried animal blood, blood meal, feather meal, egg-shell meal, hydrolyzed whole poultry, hydrolyzed hair, bone marrow, animal digest from dead, dying, diseased, or disabled animals including deer and elk, dried ruminant waste, dried swine waste, dried poultry litter, and undried processed animal waste products, fish meal, fish residue meal, crab meal, shrimp meal, fish oil, fish liver and glandular meal, fish by-products, animal fat, vegetable fat or oil, and hydrolyzed fats, edible food waste from restaurants, bakeries, and cafeterias, tetracyclines, macrolides, fluoroquinolones, streptogramins, spent mycelium and fermentation products, roxarson, arsanilic acid, copper compounds, metal amino acid complexes, urea, ammonium chloride, and ammonium sulfate, bone charcoal, calcium carbonate, chalk rock, iron salts, magnesium salts, oyster shell flour, Vitamin A, Vitamin D, Vitamin B12, Vitamin E, niacin, betaine, Aspergillis niger, Bacillus subtilis, Bifidobacterium animalis, Enterococcus faecium, yeast, aloe vera gel concentrate, ginger, capsicum, fennel, phytase, cellulase, lactase, lipase, pepsin, catalase, acetic acid, sulfuric acid, aluminum salts, dextrans, glycerin, beeswax, sorbitol, riboflavin, butylated hydroxyanisole (BHA), sodium bisulfite, herbal products, botanical products and polyethylene roughage replacement.

In one or more aspects, an FIA is included in a feed composition provided to fish as a source of nutrition. A fish feed composition is used to provide a complete diet typically containing from about 18 weight percent (wt. %) to about 50 wt. % proteins; from about 10 wt. % to about 25 wt % lipids, from about 15 wt. % to about 20 wt. % carbohydrates; from about 15 wt % to about 20 wt. % ash; less than about 8.5 wt % phosphorus; less than about 1.5 wt. % water; and less than about 10 wt. % of vitamins and minerals based on the total weight of the feed. Nonlimiting examples of ingredients in a typical fish feed composition include fish meal, fish oil, rice bran, shrimp ban, wheat bran, soybean cake, cottonseed cake, and dried potatoes.

A wide variety of commercial fish foods may be used in combination with an FIA of the present disclosure. For example, the FIA may be used in combination with and/or as a component of a pelletized feed composition, an example of which includes “BioDiet BROOD” pellets (Bioproducts, Inc., Warrenton, Oreg.). In some aspects, the FIA is included in a feed composition comprising fish meal, krill (Euphausia pacifica), cooked hydrolyzed fish, blood meal, fish oil, lecithin, vitamin A acetate, vitamin D3 supplement, vitamin E (DL-α-tocopherol acetate), pantothenic acid, niacin, inositol, vitamin B2 (riboflavin), vitamin B6(pyridoxine hydrochloride), thiamine mononitrate, folic acid, biotin, vitamin B12 supplement, processed grain byproducts, guar gum, propylene glycol, choline chloride, ascorbic acid (vitamin C), zinc sulfate, manganese sulfate, ethylene diamine dihydroiodide, and copper sulfate, potassium sorbate (a preservative), canthaxanthin, and ethoxyquin (a preservative).

In another example, the FIA may be used in combination with or as a component of a feed composition such as 40% Bass Food. In some aspects, the FIA is included in a feed composition comprising anchovy fish meal, ground wheat, soybean meal, wheat flour, meat and bone meal, fish oil, wheat bran, feather meal, dried distillers, solubles and grains, soybean oil, vitamin C (AsPP L-ascorby-2-polyphosphate), propionic acid (a mold inhibitor), DL-methionine, choline chloride, vitamin A acetate, folic acid, vitamin D3, ethoxyquin (a preservative), niacin supplement, iron sulfate, manganous oxide, vitamin E acetate, DL-calcium pantothenic acid, zinc oxide, riboflavin supplement, thiamine mononitrate, pyridoxine hydrochloride, vitamin K (menadione sodium bisulfite complex), copper oxide, biotin, vitamin B12 supplement, and sodium selenite.

In yet another example, the FIA may be used in combination with or as a component of Catfish Food. In some aspects, the FIA is included in a feed comprising soybean meal, ground wheat, wheat bran with ground wheat screenings, anchovy fish meal, wheat flour, meat and bone meal, fish oil, blood meal (spray dried), vitamin C (AsPP L-ascorbyl-2-polyphosphate), limestone meal, soybean oil, salt, choline chloride, vitamin A acetate, propionic acid (mold inhibitor), vitamin D3, niacin supplement, zinc oxide, vitamin E acetate, DL-calcium pantothenic acid, manganous oxide, iron sulfate, riboflavin supplement, vitamin B12 supplement, thiamine mononitrate, vitamin K (menadione sodium bisulfite complex), pyridoxine hydrochloride, folic acid, copper sulfate, ethylene diamine dihydroiodide, ethoxyquin (a preservative), biotin, cobalt sulfate, and sodium selenite.

The FIA may be included in the feed compositions disclosed herein in any suitable amount. The FIA may be present in the feed composition in an amount of from about 0.001 wt. % to about 5 wt. %, alternatively from about 0.1 wt. % to about 1 wt. %, alternatively from about 0.001 wt % to about 0.5 wt. % or alternatively from about 1 wt. % to about 5 wt % based on the total weight of the composition.

In some aspects, inclusion of an FIA in a feed composition of the type disclosed herein results in a reduction in phosphate discharge by the organisms ingesting the feed composition. Discharge of phosphate through urine and fecal matter may be reduced by from about 6.5 parts per million (ppm) to about 3.5 ppm, alternatively from about 3.35 ppm to about 3.2 ppm or alternatively from about 16 mg/g dry weight to about 12 mg/g dry weight when compared to an organism feed an identical composition in the absence of an FIA of the type disclosed herein. Additionally or alternatively, the reduction in the discharge of phosphate ranges from about 10% to about 50%, alternatively from about 10% to about 40% or alternatively from about 10% to about 25% in comparison to an animal feed a diet lacking a FIA of the type disclosed herein.

Additionally or alternatively, in some aspects of the present disclosure, the FIA is a multifunctional additive effective to enhance food digestion as evidenced by improved processing of phosphorous and minerals, functions to inhibit or eradicate microorganisms as indicated by antibacterial activity and/or disinfectant properties and may improve the survival rate, final weight and health of the organisms. In one or more aspects, a population of animals given a feed composition comprising a FIA of the type disclosed herein may display an overall survival rate that is increased by from about 10% to about 100%, alternatively from about 25% to about 100%, additionally or alternatively from about 50% to about 100% or alternatively greater than about 25% when compared to a population the same animals given a feed composition lacking an FIA. In one or more aspects, a population of animals given a feed composition comprising a FIA of the type disclosed herein may display a final weight that is increased by from about 10% to about 100%, additionally or alternatively from about 25% to about 100%, alternatively from about 50% to about 100% or, additionally or alternatively, greater than about 25% when compared to a population the same animals given a feed composition lacking an FIA. Herein, the final weight refers to the weight of usable portions obtained after processing.

In one or more aspects, a population of animals provided a feed composition is assessed with respect to various physiological parameters prior to provision of the feed composition and at some time period following the provision of the feed composition. For example, the population of animals may be assessed by any number of methods including determination of animal weight, survey of major organ function, determining the composition of body fluids or waste matter, and the like.

In some aspects, FIAs of the present disclosure address challenges associated with aquaculture farming such as phosphate discharge and animal health while having a low carbon footprint. In another aspect, inclusion of FIAs of the present into feed improves the nutritional value of the feed.

### Additional Disclosure

The following enumerated aspects of the present disclosures are provided as non-limiting examples.

A first aspect which is a composition comprising a sugar oxidation product and at least one material selected from the group consisting of oxides, nitrates, nitrites, sulfates, minerals, proteins, amino acids, macronutrients, and micronutrients.

A second aspect which is the composition of the first aspect wherein the sugar oxidation product comprises aldaric acid, uronic acid, glucaric acid, gluconic acid, glucuronic acid, carboxylic acids, glucose oxidation products, gluconic acid oxidation products, disaccharides, oxidized disaccharides, n-keto-acids, C2-C6 diacids, galactonic acid, galactaric acid, glutamic acid, glucodialdose, 2-ketoglucose, glucodiamine, glycolaldehyde, glyoxal, salts thereof, lactones thereof or combinations thereof.

A third aspect which is the composition of any of the first through second aspects wherein the sugar oxidation product comprises less than about 5 wt % maltose, maltotriose, fructose, higher molecular weight polysaccharides, oxidation products thereof or combinations thereof based on the total weight of the sugar oxidation product.

A fourth aspect which is the composition of any of the first through third aspects wherein the sugar oxidation product comprises gluconic acid, glucaric acid, salts thereof, derivatives thereof of or combinations thereof.

A fifth aspect which is the composition of any of the first through fourth aspects, wherein the sugar oxidation product is present in an amount of from about 0.001 wt % to about 5 wt % based on the total weight of the composition.

A sixth aspect which is the composition of any of the first through fifth aspects further comprising fish meal, fish oil, rice bran, shrimp bran, wheat bran, soybean cake, cottonseed cake, dried potatoes, corn, corn oil, rapeseed oil, sunflower oil, vegetable oil, palm oil, ground nut oil, cottonseed oil, cocoa butter oil, soya oil, or combinations thereof.

A seventh aspect which is the composition of any of the first through sixth aspects further comprising alfalfa meal, hay, Bermuda coastal grass hay, com plant, soybean hay, barley, corn, oats, rice, sorghum, wheat, canola meal, cottonseed cakes and meals, peanut meal, safflower meal, soybean (organic and genetically modified) feed and meal, distillers products, brewers dried grains, com gluten, sorghum germ cake and meal, peanut skins, wheat bran, dried citrus pulp, apple pomace, pectin pulp, beet, citrus, starch, cane molasses, meat meal, meat meal tankage, meat and bone meal, poultry meal, animal by-product meal, dried animal blood, blood meal, feather meal, egg-shell meal, hydrolyzed whole poultry, hydrolyzed hair, bone marrow; animal digest from dead, dying, diseased, or disabled animals including deer and elk; dried ruminant waste, dried swine waste, dried poultry litter, undried processed animal waste products, fish meal, fish residue meal, crab meal, shrimp meal, fish oil, fish liver and glandular meal, fish by-products, animal fat, vegetable fat or oil, and hydrolyzed fats, edible food waste from restaurants, bakeries, and cafeterias, tetracyclines, macrolides, fluoroquinolones, streptogramins, spent mycelium and fermentation products, roxarson, arsanilic acid, copper compounds, metal amino acid complexes, urea, ammonium chloride, ammonium sulfate, bone charcoal, calcium carbonate, chalk rock, iron salts, magnesium salts, oyster shell flour, Vitamin A, Vitamin D, Vitamin B12, Vitamin E, niacin, betaine, Aspergillis niger, Bacillus subtilis, Bifidobacterium animalis, Enterococcus faecium, yeast, aloe vera gel concentrate, ginger, capsicum, fennel, phytase, cellulase, lactase, lipase, pepsin, catalase, acetic acid, sulfuric acid, aluminum salts, dextrans, glycerin, beeswax, sorbitol, riboflavin, butylated hydroxyanisole (BHA), sodium bisulfite, herbal products, botanical products, polyethylene roughage replacement or a combination thereof.

An eighth aspect which is a method comprising providing an animal a composition comprising a sugar oxidation product and at least one material selected from the group consisting of oxides, nitrates, nitrites, sulfates, minerals, proteins, amino acids, macronutrients, and micronutrients.

A ninth aspect which is the method of the eighth aspect wherein the animal is selected from the group consisting of bovinae, cervidae, lagomorphs, oves, suidae birds, fish, crustaceans, and mollusks.

A tenth aspect which is the method of any of the eighth through ninth aspects wherein the animal is a fish, crustacean, or mollusk.

An eleventh aspect which is the method of any of the eighth through tenth aspects further comprising assessing the physiology of the animal at some time period subsequent to providing the compositions.

A twelfth aspect which is the method of the eleventh aspect wherein the animal displays (i) an increased total weight, (ii) a reduced phosphorous discharge, (iii) an increased survival rate or health benefit, (iv) increased feed conversion ratio, or (v) a combination thereof.

A thirteenth aspect which is the method of the eleventh aspect wherein the reduced phosphorous discharge ranges from about 10% to about 50% when compared to the phosphorous discharge of an organism provided an otherwise similar composition lacking a sugar oxidation product.

A fourteenth aspect which is the method of any of the eighth through thirteenth aspects wherein the sugar oxidation product comprises gluconic acid, glucaric acid, salts thereof, derivatives thereof of or combinations thereof.

A fifteenth aspect which is the method of any of the eighth through fourteenth aspects wherein the sugar oxidation is present in an amount of from about 0.001 wt. % to about 5 wt. % based on the total weight of the composition.

A sixteenth aspect which is a method comprising a blend of a sugar oxidation product and a mixture comprising from about 18 weight percent (wt. %) to about 50 wt. % proteins; from about 10 wt % to about 25 wt. % lipids or combinations thereof.

A seventeenth aspect which is the method of the sixteenth aspect further comprising from about 15 wt. % to about 20 wt % carbohydrates; less than about 8.5 wt. % phosphorus; less than about 1.5 wt. % water; and less than about 10 wt % of vitamins and minerals based on the total weight of the feed.

An eighteenth aspect which is the method of any of the sixteenth through seventeenth aspects wherein the mixture further comprises one or more fatty acids.

A nineteenth aspect which is the method of the eighteenth aspect wherein the fatty acids comprise omega-3 fatty acids, omega-6 fatty acids or combinations thereof.

A twentieth aspect which is the method of any of the sixteenth through nineteenth aspects wherein the mixture further comprises calcium, sodium, chloride, potassium, chlorine, sulfur, phosphorous, magnesium, iron, copper, chromium, iodine, manganese, zinc, selenium or combinations thereof.

A twenty-first aspect which is the method of any of the sixteenth through twentieth aspects wherein the sugar oxidation product comprises gluconic acid, glucaric acid, salts thereof, derivatives thereof of or combinations thereof.

### EXAMPLE

The aspects having been generally described, the following example is given as particular aspects of the disclosure and to demonstrate the practice and advantages thereof. It is understood that the example is given by way of illustration and is not intended to limit the specification or the claims in any manner.

### Example 1

To validate if gluconates can be used as an acidulant additives in fish feed to increase shrimp's digestion and reduce phosphate discharge to aquaculture farm water, freshwater shrimp were fed with 1) regular shrimp diet as a control, 2) the same shrimp diet with 1.2% of an FIA, particularly gluconate, as disclosed herein and 3) the shrimp diet with 1.2% sodium citrate as an additive.

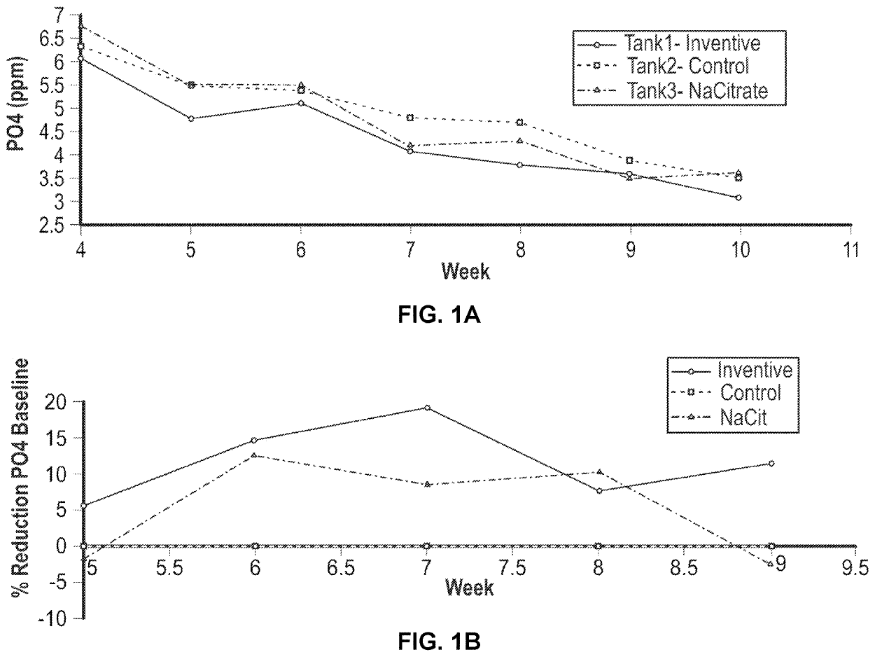

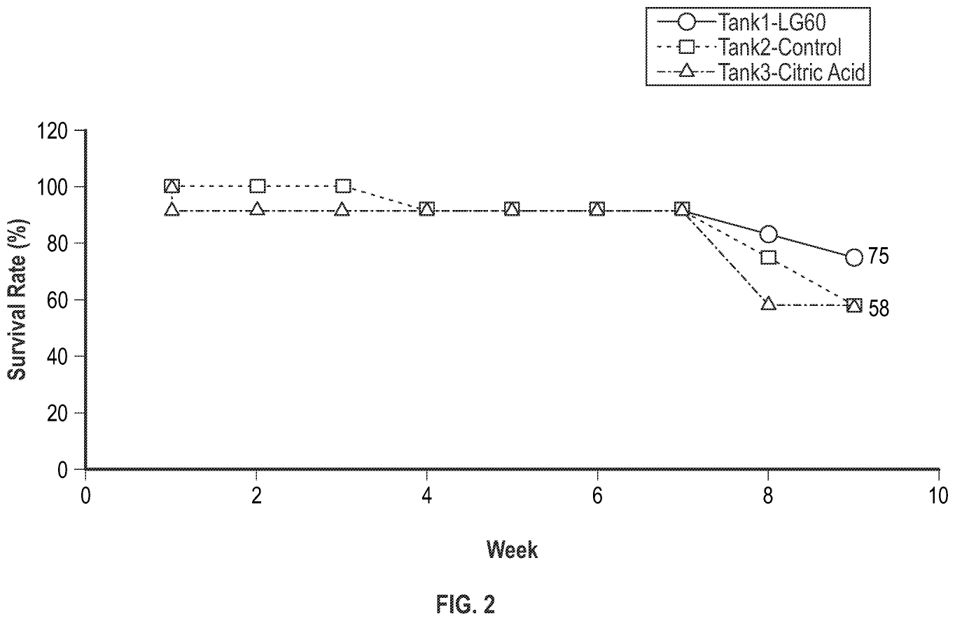

Phosphate reduction was observed in the tank with shrimp fed the gluconate added diet (up to 19 wt. %) when compared to control (regular shrimp diet). Phosphate reduction was equivalently high level to slightly better than citric acid up to 12.5 wt. %. This functionality of reducing phosphate level in discharged water from aquaculture farms is beneficial with respect to environmental regulations as aquaculture farmers are dealing with and the restrictions on the allowed phosphate level in the discharged water waste from aquaculture farms. The results are presented in FIGS. 1A and 1B.

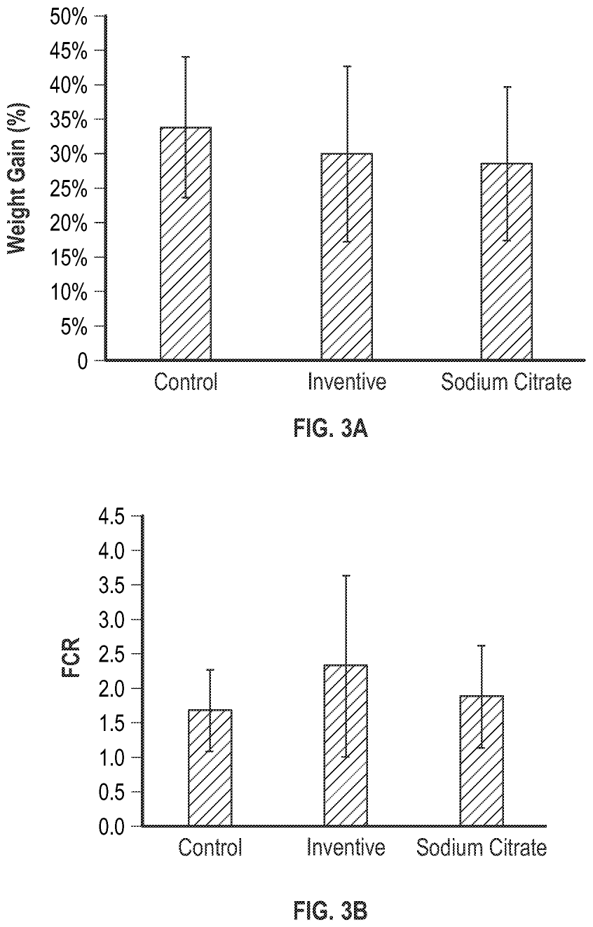

In addition, higher survival rates were observed with the shrimps fed with a FIA (75%) compared to a citric acid additive and regular shrimp diet (58%) over the course of 10 weeks of feeding trial. The results are presented in FIG. 2.

### Example 2

The efficacy of an FIA used as fish feed was investigated. The aim of this study was to assess the efficacy of (i) a feed comprising an FIA of the type disclosed herein (e.g., gluconate) on the growth, phosphorus discharge, biochemical composition and histopathology in tilapia when compared to (ii) a soybean meal-based diet that would have a similar formulation as commercial feeds or (iii) a soybean meal-based diet that includes an FIA of the type disclosed herein or sodium citrate, respectively. Sodium citrate was chosen because this is a competitive chelator and has in the past shown to improve phosphorus utilization/reduce phosphorus discharge in various aquatic animals.

To compare the efficacy of an FIA of the type disclosed herein, a total of 4 isonitrogenous and isolipidic diets were formulated to satisfy the nutritional requirements of tilapia. The crude protein and crude lipid content of the diets were all 32 wt. % and 8 wt. %, respectively. However, the percentages of proteins and supplements were altered to satisfy the aims of this study. Soybean meal was used across all diets. Diet 1 was the control with only soybean meal (cellulose at 0.5 wt. %). Diet 2 had an FIA, gluconate, at 0.5 wt. %, Diet 3 had sodium citrate at 0.5 wt %.

The FIA appeared like packaging peanuts, and thus needed to be ground into a fine powder for the product to be adequately mixed in the diet. All diets were made using a pelleter and the pellets were dried in an oven at 60° C. and stored at −20° C. until use.

A total of 90 all male tilapia were equally distributed among 9 oval polyethylene tanks that ran on a recirculating system. Thus, all the fish shared the same water throughout the 2 month growth trial. After the fish were stocked, of approximately equal size, the tanks were randomly allocated their treatments in triplicate. For the first week, the tilapia were fed the control diet to acclimate them to the experimental conditions and then after a week were hand fed twice daily to apparent satiation on their respective experimental diets.

Throughout the study, excess waste was manually siphoned out of the system and water exchanges were provided weekly. Feed intake was recorded to later calculate the feed conversion ratio. Fish feces were collected, dried and measured for the phosphorus levels after 2 months.

After 2 months, all the fish were weighed to assess weight gain. Two fish/tank were sampled for liver and intestinal histopathology and the muscle sent out for biochemical analysis. The remaining fish were placed in new clean water and the water was sampled at 4 hours and measured for phosphorus levels mostly derived from urine.

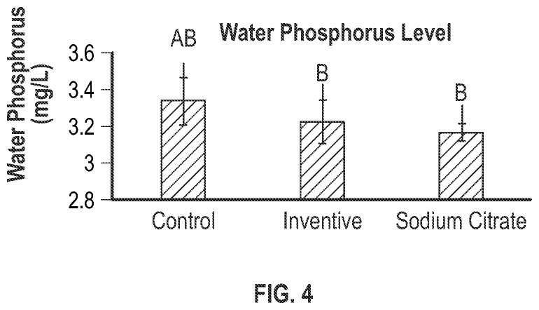

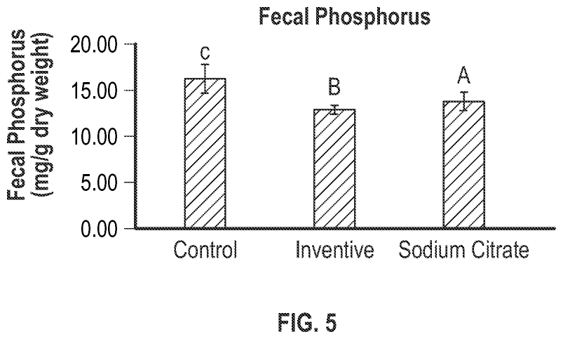

In terms of fish growth, there were no significant differences in growth rates across the diets after 2 months. The results are presented in FIG. 3. The supplements had no effect on palatability, based on the feed intake being similar, while the feed conversion ratio were similar across all dietary treatments. On the other hand, fish fed with an FIA of the type disclosed herein and citrate additives had significantly less phosphorus discharge (via urine) in the water (after 4 hours) compared to those fed the soybean based control diet. (FIG. 4).

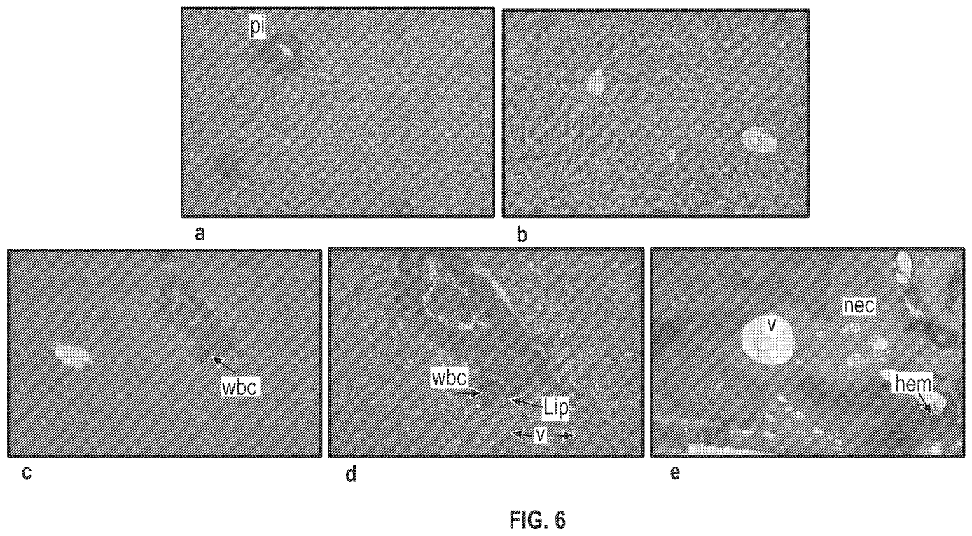

Similar results were observed in terms of the phosphorus level in the fecal matter after feeding the diets for 2 months. (FIG. 5) The sodium citrate supplement is known to enhance phytase activity in tilapia as well as being a competitive chelator which may have contributed to less phosphorus discharge via the urine of fish. This finding was further supported by measuring the fecal phosphorus levels after 2 months, which showed significantly less phosphorus content from fish fed diets with an FIA of the type disclosed herein and citrate supplements compared to the one fed with control diets.

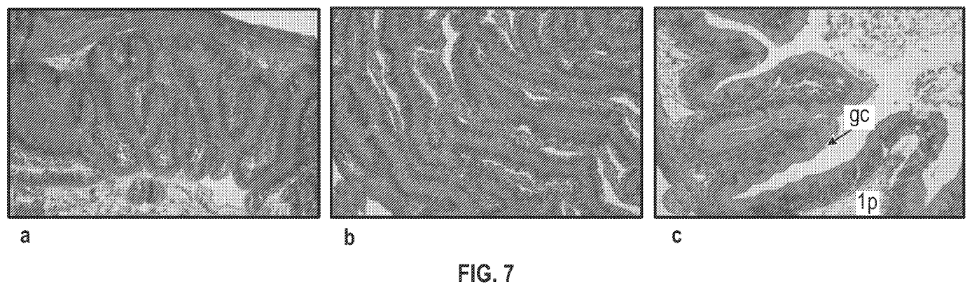

Although diets containing an FIA of the type disclosed herein and citrate additive provided similar results in terms of growth and phosphorus discharge/utilization, the histopathology showed some crucial differences (FIG. 6). First, an FIA of the type disclosed herein showed no hepatic toxicity to fish (FIG. 6b). The lack of hepatic toxicity is a surprisingly advantageous result as many dietary supplements may display short-term benefits but are deleterious in terms of organ toxicity in the long term. Sodium citrate was found to result in hepatic toxicity based on both mild effects (localized inflammation) to severe (extensive necrosis, vacuolization and hemorrhaging) which may result in health problems as the animals grow. Moreover, fish fed the diet with sodium citrate showed some pathologies that included inflammation of the intestinal villi and goblet cell hyperplasia (FIG. 7c). These histopathologies indicate that sodium citrate acted as an irritant to the tilapia intestine but no such adverse effects were observed in tilapia fed diets with an FIA of the type disclosed herein and control. Consequently, the livers in tilapia fed control and diets containing an FIA of the present disclosure showed normal sinusoid organization with clear central veins and (b) intact and well dispersed pancreatic islets (c). However, tilapia fed with sodium citrate had varying degrees of hepatic lesions that included white blood cell (wbc) infiltrations and presence of lipofuscin-like material (lip) that are consistent with inflammation as well as more severe pathologies that included necrosis (nec) leading to the presence of plasma throughout the liver, hemorraghing (hem) and extensive vacuolization (v). ×10 magnification (b, c, d, e) and ×20 magnification (d). The livers in tilapia fed citrate diet showed severe inflammation in goblet cell (gc) and the lamina propria (Ip) where there was either some inflammation or a disintegration of the architecture.

Overall, results indicate that an FIA of the type disclosed herein, at the level tested, was safe for tilapia consumption and significantly reduced phosphorus discharge in both the urine and feces.

While preferred embodiments have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the disclosure. For example, the relative dimensions of various parts, the materials from which the various parts are made, and other parameters can be varied. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

Each and every claim is incorporated into the specification as an aspect of the present disclosure. Thus, the claims are a further description and are an addition to the aspects of the present invention. The discussion of a reference herein is not an admission that it is prior art to the presently disclosed subject matter, especially any reference that may have a publication date after the priority date of this application. The disclosures of all patents, patent applications, and publications cited herein are hereby incorporated by reference, to the extent that they provide exemplary, procedural or other details supplementary to those set forth herein.

1. A composition, comprising:
a sugar oxidation product; and
at least one material selected from the group consisting of oxides, nitrates, nitrites, sulfates, minerals, proteins, amino acids, macronutrients, and micronutrients.

2. The composition of claim 1, wherein the sugar oxidation product comprises aldaric acid, uronic acid, glucaric acid, gluconic acid, glucuronic acid, carboxylic acids, glucose oxidation products, gluconic acid oxidation products, disaccharides, oxidized disaccharides, n-keto-acids, C2-C6 diacids, galactonic acid, galactaric acid, glutamic acid, glucodialdose, 2-ketoglucose, glucodiamine, glycolaldehyde, glyoxal, salts thereof, lactones thereof or combinations thereof.

3. The composition of claim 1, wherein the sugar oxidation product comprises less than about 5 wt. % maltose, maltotriose, fructose, higher molecular weight polysaccharides, oxidation products thereof or combinations thereof based on the total weight of the sugar oxidation product.

4. The composition of claim 1, wherein the sugar oxidation product comprises gluconic acid, glucaric acid, salts thereof, derivatives thereof of or combinations thereof.

5. The composition of claim 1, wherein the sugar oxidation product is present in an amount of from about 0.001 wt. % to about 5 wt % based on the total weight of the composition.

6. The composition of claim 1, further comprising fish meal, fish oil, rice bran, shrimp bran, wheat bran, soybean cake, cottonseed cake, dried potatoes, com, com oil, rapeseed oil, sunflower oil, vegetable oil, palm oil, ground nut oil, cottonseed oil, cocoa butter oil, soya oil, or combinations thereof.

7. The composition of claim 1, further comprising alfalfa meal, hay, Bermuda coastal grass hay, corn plant, soybean hay, barley, corn, oats, rice, sorghum, wheat, canola meal, cottonseed cakes and meals, peanut meal, safflower meal, soybean (organic and genetically modified) feed and meal, distillers products, brewers dried grains, com gluten, sorghum germ cake and meal, peanut skins, wheat bran, dried citrus pulp, apple pomace, pectin pulp, beet, citrus, starch, cane molasses, meat meal, meat meal tankage, meat and bone meal, poultry meal, animal by-product meal, dried animal blood, blood meal, feather meal, egg-shell meal, hydrolyzed whole poultry, hydrolyzed hair, bone marrow; animal digest from dead, dying, diseased, or disabled animals including deer and elk; dried ruminant waste, dried swine waste, dried poultry litter, undried processed animal waste products, fish meal, fish residue meal, crab meal, shrimp meal, fish oil, fish liver and glandular meal, fish by-products, animal fat, vegetable fat or oil, and hydrolyzed fats, edible food waste from restaurants, bakeries, and cafeterias, tetracyclines, macrolides, fluoroquinolones, streptogramins, spent mycelium and fermentation products, roxarson, arsanilic acid, copper compounds, metal amino acid complexes, urea, ammonium chloride, ammonium sulfate, bone charcoal, calcium carbonate, chalk rock, iron salts, magnesium salts, oyster shell flour, Vitamin A, Vitamin D, Vitamin B12, Vitamin E, niacin, betaine, Aspergillis niger, Bacillus subtilis, Bifidobacterium animalis, Enterococcus faecium, yeast, aloe vera gel concentrate, ginger, capsicum, fennel, phytase, cellulase, lactase, lipase, pepsin, catalase, acetic acid, sulfuric acid, aluminum salts, dextrans, glycerin, beeswax, sorbitol, riboflavin, butylated hydroxyanisole (BHA), sodium bisulfite, herbal products, botanical products, polyethylene roughage replacement or a combination thereof.

8. A method, comprising:
providing an animal a composition comprising a sugar oxidation product and at least one material selected from the group consisting of oxides, nitrates, nitrites, sulfates, minerals, proteins, amino acids, macronutrients, and micronutrients.

9. The method of claim 8, wherein the animal is selected from the group consisting of bovinae, cervidae, lagomorphs, oves, suidae birds, fish, crustaceans, and mollusks.

10. The method of claim 8, wherein the animal is a fish, crustacean, or mollusk.

11. The method of claim 8, further comprising assessing the physiology of the animal at some time period subsequent to providing the compositions.

12. The method of claim 11, wherein the animal displays (i) an increased total weight, (ii) a reduced phosphorous discharge, (iii) an increased survival rate or health benefit, (iv) increased feed conversion ratio, or (v) a combination thereof.

13. The method of claim 11, wherein the reduced phosphorous discharge ranges from about 10% to about 50% when compared to the phosphorous discharge of an organism provided an otherwise similar composition lacking a sugar oxidation product.

14. The method of claim 8, wherein the sugar oxidation product comprises gluconic acid, glucaric acid, salts thereof, derivatives thereof of or combinations thereof.

15. The method of claim 8, wherein the sugar oxidation is present in an amount of from about 0.001 wt. % to about 5 wt. % based on the total weight of the composition.

16. A composition, comprising:
a blend of a sugar oxidation product and a mixture comprising from about 18 weight percent (wt %) to about 50 wt. % proteins, from about 10 wt % to about 25 wt. % lipids, or combinations thereof.

17. The composition of claim 16, further comprising from about 15 wt % to about 20 wt. % carbohydrates; less than about 8.5 wt. % phosphorus; less than about 1.5 wt. % water; and less than about 10 wt. % of vitamins and minerals based on the total weight of the feed.

18. The composition of claim 16, further comprising one or more fatty acids.

19. The composition of claim 18, wherein the fatty acids comprise omega-3 fatty acids, omega-6 fatty acids or combinations thereof.

20. The composition of claim 18, further comprising calcium, sodium, chloride, potassium, chlorine, sulfur, phosphorous, magnesium, iron, copper, chromium, iodine, manganese, zinc, selenium or combinations thereof.

21. The composition of claim 16, wherein the sugar oxidation product comprises gluconic acid, glucaric acid, salts thereof, derivatives thereof of or combinations thereof.
